(19) United States
(12) Patent Application Publication
(10) Pub. No.: US 2025/0312623 A1
(43) Pub. Date: Oct. 9, 2025

(54) CONFORMAL PHASED-ARRAY TRANSDUCER ARRANGEMENT

(71) Applicant: Insightec, Ltd., Tirat Carmel (IL)

(72) Inventors: Yoav LEVY, Hinanit (IL); Jacob VORTMAN, Tirat Carmel (IL); Adi GREENBERG, Haifa (IL)

(21) Appl. No.: 18/842,392

(22) PCT Filed: Feb. 7, 2023
(86) PCT No.: PCT/IB2023/051089

§ 371 Date: Aug. 29, 2024

Related U.S. Application Data
(60) Provisional application No. 63/307,958, filed on Feb. 8, 2022.
(60) Provisional application No. 63/482,054, filed on Jan. 29, 2023.

(51) Int. Cl. A61N 7/02; A61N 7/00; G16H 40/63

(52) U.S. Cl. CPC A61N 7/02; G16H 40/63; A61N 2007/0078; A61N 2007/0091

(57) ABSTRACT

Conformal ultrasound transducer arrangements and supporting registration systems and methods enable automated mapping of transducer elements to the internal anatomy of a patient. Once established, the mapping remains valid despite patient movements, and can be used to select transducer elements and their relative phases so as to create a high-energy focus at an internal target of interest without damage to intervening non-target tissue.

### FIELD OF THE INVENTION

The present invention relates, in general, to ultrasound therapy, and, in particular, to systems and methods for delivering targeted ultrasound therapy to internal anatomic regions.

### BACKGROUND

Tissue, such as a benign or malignant tumor, organ, or other body region may be treated invasively by surgically removing the tissue, or with minimal intrusion or fully non-invasively by using, for example, thermal ablation. Both approaches may effectively treat certain localized conditions, but involve delicate procedures to avoid destroying or damaging otherwise healthy tissue.

Thermal ablation, as may be accomplished using focused ultrasound, has particular appeal for treating diseased tissue surrounded by or neighboring healthy tissue or organs because the effects of ultrasound energy can be confined to a well-defined target region. Ultrasonic energy may be focused to a zone having a cross-section of only a few millimeters due to relatively short wavelengths (e.g., as small as 1.5 millimeters (mm) in cross-section at one Megahertz (1 MHz)). Moreover, because acoustic energy generally penetrates well through soft tissues, intervening anatomy often does not impose an obstacle to defining a desired focal zone. Thus, ultrasonic energy may be focused at a small target in order to ablate diseased tissue while minimizing damage to surrounding healthy tissue. If the target volume is larger than the focus, the focus may be moved until it is fully within the target volume.

To focus ultrasonic energy at a desired target, drive signals may be sent to an acoustic transducer having a number of transducer elements such that constructive interference occurs at the focal zone. At the target, sufficient acoustic intensity may be delivered to heat tissue until necrosis occurs, i.e., until the tissue is destroyed. Preferably, non-target tissue along the path through which the acoustic energy propagates (the “pass zone”) outside the focal zone is exposed to low-intensity acoustic beams and thus will be heated only minimally, if at all, thereby minimizing damage to tissue outside the focal zone.

FIG. 1 illustrates a known ultrasound system 100 for focusing ultrasound onto a target region 101 through the skull. The system 100 includes a phased array 102 of transducer elements 104, a beamformer 106 driving the phased array 102, a controller 108 in communication with the beamformer 106, and a frequency generator 110 providing an input electronic signal to the beamformer 106.

The array 102 may have a curved (e.g., spherical or parabolic) shape suitable for placing it on or near (e.g., separated by a water-filled pad or other materials that are acoustically transparent) the surface of the skull or a body part other than the skull, or may include one or more planar or otherwise shaped sections. Its dimensions may vary, depending on the application, between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials capable of converting electrical energy to acoustic energy, may also be used. To assure maximum power transfer to the transducer elements 104, the elements 104 may be configured for electrical resonance at 50 Ω matching input connector impedance.

The transducer array 102 is coupled to the beamformer 106, which drives the individual transducer elements 104 so that they collectively produce a focused ultrasonic beam or field. For n transducer elements, the beamformer 106 may contain n driver circuits, each circuit including or consisting of an amplifier 118 and a phase delay circuit 120; drive circuit drives one of the transducer elements 104. The beamformer 106 receives a radio frequency (RF) input signal, typically in the range from 0.1 MHz to 10 MHz, from the frequency generator 110, which may, for example, be a Model DS345 generator available from Stanford Research Systems. The input signal may be split into n channels for the n amplifiers 118 and delay circuits 120 of the beamformer 106. The radio frequency generator 110 and the beamformer 106 drive the individual transducer elements 104 of the transducer array 102 at the same frequency, but at different phases and/or different amplitudes such that the transducer elements 104 collectively form a “phased array.”

The acoustic waves/pulses transmitted from the transducer elements 104 form an acoustic energy beam, and the transducer elements are driven so that the waves/pulses converge at a focal zone in the targeted tissue 101. Within the focal zone, the wave energy of the beam is (at least partially) absorbed by the tissue, thereby generating heat, cavitation and/or mechanical forces and raising the temperature of the tissue for therapeutic and/or diagnostic purposes. For example, the tissue may be heated to a point where the cells are denatured and/or ablated. To effectively treat the target tissue, the acoustic energy beam must be precisely focused to the target location 101 to avoid damage to healthy tissue surrounding the target region. Parameters (e.g., the phase shifts a₁-aₙ and/or amplification or attenuation factors α₁-αₙ) of the drive signals associated with the transducer elements may be adjusted so as to compensate for acoustic aberrations and thereby improve focusing properties at the target region 101.

Generally, the amplification factors and phase shifts may be computed using the controller 108, which may determine the parameters based on information about the characteristics (e.g., structure, thickness, density, etc.) of the skull and their effects on propagation of acoustic energy. For example, such information may be obtained from an imager 112. The amplification factors and phase shifts may be computed using the controller 108.

Because placement of the transducer array 102 on the patient is somewhat arbitrary, the clinician attempts to position the array so that, based on rough knowledge of the target location, it is geometrically possible to generate a focal point at the target 101. If the transducer array 102 is not directly secured to the patient's head (or other body part), it is generally necessary to track relative movement between the patient's head and the transducer array 102 so that the focus is maintained at the target 101. But even if the transducer array 102 is directly secured to the patient, it is still necessary to computationally establish a mapping among the coordinate reference frames of the transducer array 102, the imager 112, and the patient's anatomy, which can be time-consuming and inconvenient.

### SUMMARY

The present invention provides conformal transducer arrangements and supporting registration systems and methods that enable automated mapping of transducer elements to the internal anatomy of a patient. Once established, the mapping remains valid despite patient movements, and can be used to select transducer elements and their relative phases so as to create a high-energy focus at an internal target of interest without damage to intervening non- target tissue.

Systems in accordance herewith may facilitate positioning transducer elements volumetrically in an optimal way so as to enhance efficacy, including improved treatment rates and reduced adverse events.

Accordingly, in a first aspect, the invention pertains to a transducer arrangement comprising, in various embodiments, a flexible, conformal scaffold shaped to fit over a portion of a patient's anatomy; a plurality of ultrasound transducer elements movably positioned on the conformal scaffold; and over at least some of the transducer elements, a visible fiducial element identifying the transducer element and visible with the conformal scaffold positioned on the patient. In various embodiments, the transducer arrangement has only one fiducial ID. In some embodiments, the transducer arrangement has one or more fiducial IDs. In some embodiments, some of the fiducial elements are unique. At least some of the fiducial elements may be uniquely identifiable based on relative position with respect to at least one of (i) the scaffold or (ii) at least one other fiducial element. In various embodiments, all of the transducer elements have associated fiducial elements. In other embodiments, only some of the plurality of transducer elements have fiducial elements and positions of transducer elements without fiducial elements are determined from positions of the some of the plurality of transducer elements. Each of the plurality of transducer element may have maximum linear dimensions no greater than 0.7λ, 1λ, 2λ, 3λ, or 5λ, where λ is an emission wavelength of the ultrasound transducer elements.

In some implementations, the plurality of transducer elements and their associated fiducial elements are configured for mobility along the conformable scaffold. The fiducial elements may be rectangular. The fiducial elements are 2D barcodes or visual markers (e.g., April tag, ArUco Tag, etc.) In various embodiments, the conformal scaffold is made of a breathable fabric. Alternatively, the conformal scaffold may include a plurality of conformal frame elements that slidably carry the plurality of transducer elements. The transducer elements may be pivotable about the conformal frame elements. In various embodiments, the conformal scaffold includes a grid of movably linked frame elements, at least some of the frame elements carrying the plurality of transducer elements.

In some embodiments, each of the plurality of the transducer elements comprises an emission element and a flexible container for a coupling liquid. The conformal scaffold may permit injection therethrough of ultrasound coupling gel.

In another aspect, the invention relates to a system for generating an ultrasound focus at a target region. In various embodiments, the system comprises a flexible, conformal scaffold shaped to fit over a portion of a patient's anatomy; a plurality of transducer elements movably positioned on the conformal scaffold; over at least some of the plurality of transducer elements, a unique, visible fiducial element identifying the transducer element and visible with the conformal scaffold positioned on the patient; a plurality of sensors for acquiring images of the conformal scaffold; and a controller, operably coupled to the plurality of transducer elements and the computer vision system, configured to (a) computationally analyze the acquired images and, based thereon, establish positions and orientations of the fiducial elements in a spatial coordinate system; and (b) based on the established positions and orientations, operate at least some of the plurality of transducer elements to collectively transmit an ultrasound beam to a target region in the spatial coordinate system.

In some embodiments, the plurality of transducer elements and their associated fiducial elements are configured for mobility along the conformable scaffold. The controller may be configured to represent the fiducial elements and the target region in a common spatial reference frame. Some of the fiducial elements may be unique, or at least some of the fiducial elements may be uniquely identifiable by the controller based on relative position, in the sensor images, with respect to the scaffold and/or at least one other fiducial element.

In various embodiments, all of the plurality of transducer elements have associated fiducial elements. In other embodiments, only some of the plurality of transducer elements have the fiducial elements and positions of transducer elements without fiducial elements are determined from positions of the some of the plurality of transducer elements. Each transducer element may have maximum linear dimensions no greater than 0.7λ, 1λ, 2λ, 3λ, or 5λ, where λ is an emission wavelength of the plurality of transducer elements.

In some embodiments, the conformal scaffold is made of a breathable fabric. The conformal scaffold may include a plurality of conformal frame elements that slidably carry the transducer elements. The conformal scaffold may include a grid of movably linked frame elements, at least some of the frame elements carrying the plurality of transducer elements. In various embodiments, each of the plurality of transducer elements comprises an emission element and a flexible container for a coupling liquid.

The controller may be configured to cause movement of the plurality of transducer elements relative to the conformal scaffold to improve an ultrasound focus at the target region. For example, the movement may include sliding and pivoting.

In yet another aspect, the invention pertains to a method of generating an ultrasound focus at a target region using an ultrasound transducer array comprising a plurality of transducer elements movably positioned on a conformal scaffold. In various embodiments, the method comprises the steps of computationally establishing positions and orientations of the plurality of transducer elements and the target region in a spatial coordinate system; electronically tracking spatial positions and orientations of the plurality of transducer elements; based on the tracked positions and orientations, computationally analyzing images acquired by one or more sensors; and operating at least some of the plurality of transducer elements to collectively transmit an ultrasound beam to the target region.

In various embodiments, the method further comprises causing movement of the plurality of transducer elements along the conformable scaffold, e.g., during operation of a focusing procedure. The fiducial elements and the target region may be computationally localized in a common spatial reference frame.

In various embodiments, the plurality of transducer elements comprise trackable fiducial elements. The fiducial elements are 2D barcodes or visual markers (e.g., April tag,

ArUco Tag, etc.). The transducer elements may receive power and control signals via wires, or the plurality of transducer elements may be self-powered and responsive to wireless signals. In some embodiments, the visual markers are placed on a surface of the body of the patient as to detect a movement of the body relative to the plurality of transducer elements.

As used herein, the term “substantially” means ±10%, and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 schematically depicts a prior-art ultrasound system.

FIG. 2 schematically illustrates an ultrasound system including a conformal transducer array in accordance with embodiments of the present invention.

FIG. 3 illustrates a series of representative fiducial markers.

FIG. 4 is a schematic elevation of a representative transducer element in accordance with embodiments hereof.

FIG. 5 is a perspective view of an alternative embodiment of a conformal transducer array in accordance herewith.

FIG. 6A schematically depicts another embodiment of a conformal transducer array.

FIG. 6B is an elevational view of a transducer assembly suitable for use in connection with the conformal transducer array shown in FIG. 6A.

FIG. 7 is a perspective view of an example marker positioning on the face of a patient in accordance with some embodiments.

### DETAILED DESCRIPTION

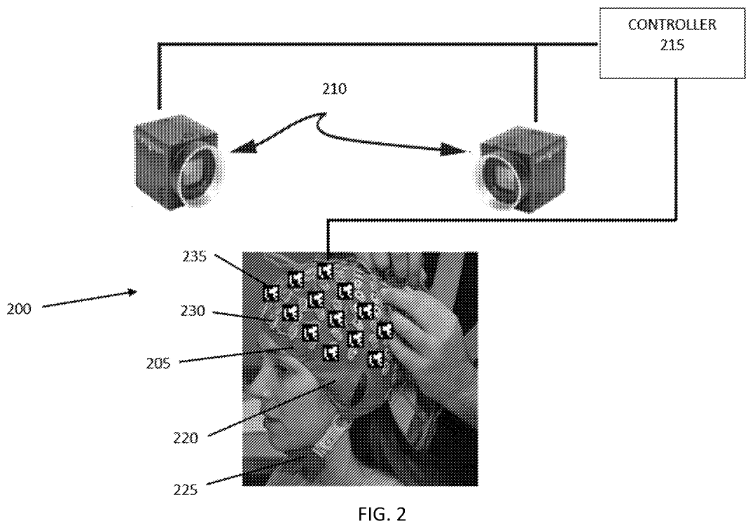

Refer first to FIG. 2, which illustrates an exemplary system 200 for focusing ultrasound onto an internal anatomic region, here within a patient's skull. One of ordinary skill in the art, however, will understand that the ultrasound system 200 described herein may be applied to any part of the human body. In various embodiments, the system 200 includes a conformal ultrasound transducer array 205, a plurality of sensors 210, and a controller 215. The transducer array 205 includes a flexible scaffold in the form of a cap 220 and an adjustable chin strap 225; in use, the cap is positioned over the patient's head and secured with the chin strap 225 to prevent movement. The cap may be made of any durable, flexible material and is desirably moisture-permeable for the patient's comfort. Cloth materials such as cotton and “breathable” elastic fabrics such as polyester, spandex and micro-mesh materials are suitable.

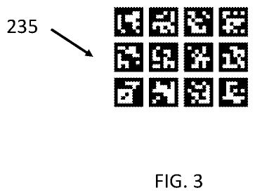

Distributed over the surface of the cap 220 is an arrangement of ultrasound transducers representatively indicated at 230. Associated with each transducer element 230 is a visible fiducial or marker 235. Each is typically unique visually as depicted in FIG. 3. (For ease of illustration, FIG. 2 does not show every marker 235.) For example, as illustrated, the markers 235 may be different 2D barcodes. Suitable barcode types include ARTag, AprilTag, and ArUco markers.

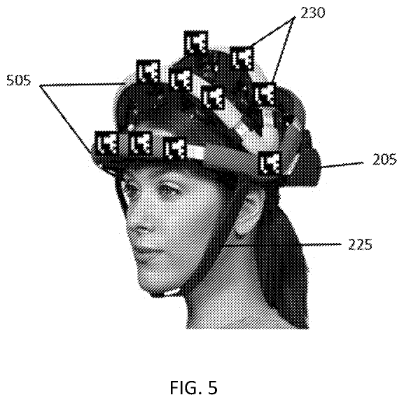

Unique fiducials are not strictly necessary, however, if other information can be used to locate a transducer element 230. For example, the scaffold 220 may be configured to constrain transducer positions in a manner that facilitates their relative localization. Thus, if a plurality of transducer elements are positionable on the scaffold along a bar that has been spatially localized in a coordinate reference frame (see FIG. 5 and discussion below), the controller 215 need only read the fiducial of one of the transducer elements and may assign identifiers to the other elements whose determined spatial locations in the coordinate reference frame correspond to allowable positions along the bar. In this way, the positions and orientations of transducer elements lacking fiducials (or with redundant fiducials—e.g., all elements along the bar may have the same fiducial 235). Moreover, if the scaffold constrains both the number and positions of transducer elements 230, it is not necessary for the controller 215 to localize each of them spatially. If, for example, the scaffold includes a bar with detents or other positioning features, the spatial locations of transducer elements therealong may be computed based on the spatial position and orientation of the bar. More generally, it should be noted that transducer position optimization may be complex depending on the number of elements, and may be carried out manually or using a suitable optimization technique; complex multiparametric optimizations may benefit from deep learning approaches.

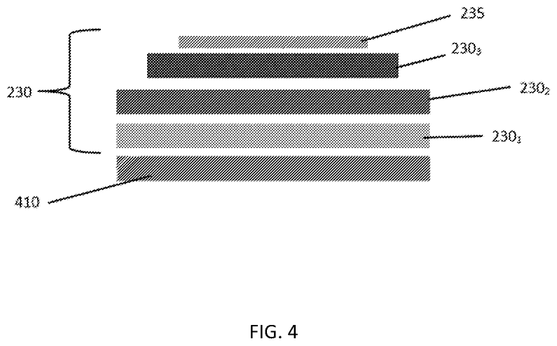

The structure of the transducer elements 230 is shown in FIG. 4. The illustrated element 230 includes a piezoelectric ceramic membrane 230₁, an overlying circuit board 230₂ and suitable electronic elements 230₃ thereon. For example, driver (amplifier) electronics may be implemented on the circuit board 230₂ so that the operating signals supplied by the controller 215 to operate the transducer elements 230 can be low power. A thin layer 410 of ultrasound coupling gel may be applied to the skin to provide direct contact between the membrane 230₁ and the skin, thereby allowing efficient transfer of ultrasound energy into the patient's body. For example, the gel 410 may be injected through openings in the cap 220. The membrane 230₁ may have maximum linear dimensions no greater than 0.7λ, 1λ, 2λ, 3λ, or 5λ (e.g., less than 0.5λ), where λ is the wavelength of the emitted ultrasound. This allows a large electronic steering envelope (e.g., ±40°) to be achieved.

The transducer elements 230 may be movable relative to the conformal scaffold 220, e.g., along guides that facilitate orientation changes to maintain perpendicularity of the transducer elements to the patient's skin. For example, in the configuration shown in FIG. 2, the elements 230 may be movable within pockets or along pleats within the fabric of the cap. Alternatively, as shown in FIG. 5, the conformal ultrasound transducer array 205 may utilize a series of frame elements 505 that carry the transducer elements 230, which are capable of slidable movement therealong. This movement may be manual or autonomous, e.g., bidirectional motors on the circuit board 230₂ may shift the positions of the various transducer elements 230 along the frames 505 as directed by the controller 215. In some embodiments, the frames 505 may extend from front to back so that the wearer's hair may be combed and gathered between the frame segments.

The transducer elements 230 (and any motive components integrated therewith) may be self-powered (e.g., the electronic elements 230₃ may include a battery) and received wireless control signals, or may be powered via cables that also provide a bidirectional control signal path.

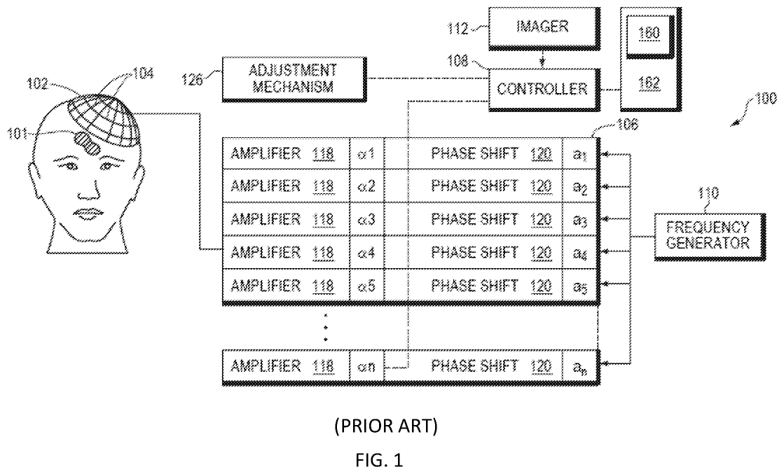

In operation, the sensors 210 are positioned around the treatment room and are sufficient in number—at least two, and typically at least three or more—that more than one sensor records all of the markers 235. The sensors 210 may be high-definition area cameras (e.g., CMOS cameras such as the BLACKFLY S GigE camera supplied by Teledyne FLIR), 3D depth cameras, time-of-flight cameras, or any other suitable digital sensor for recording high-definition images capable of resolving the different markers 235. The controller 215 includes conventional machine-vision functionality that allows it to ascertain the spatial coordinates, typically in a reference frame of the room, of each the markers 235 as well as their orientations (hence the utility of rectangular 2D barcodes as markers, since orientation can be detected easily) based on the images recorded by the sensors 210. This may be accomplished stereoscopically, based on image frames including the same marker recorded by multiple sensors 210 whose spatial positions are known, or directly using a 3D camera with the marker in its field of view. The position of the target 101 (see FIG. 1) relative to the patient's skull (and, hence, relative to the conformal transducer array 205) may be established using an imager 112, which may be a magnetic resonance (MR) imaging device, a computer tomography (CT) device, a positron emission tomography (PET) device, a single-photon emission computed tomography (SPECT) device, or an ultrasonography device. Image acquisition may be three-dimensional (3D) or, alternatively, the imager 112 may provide a set of two-dimensional (2D) images suitable for reconstructing a three-dimensional image of the target region 101 and/or other regions (e.g., the region surrounding the target 101, the region in the pass zone located between the transducer and the target, or another target region).

Based on this mapping, the controller 215 may utilize a general-purpose or special-purpose digital data processor programmed with software in a conventional manner, and without undue experimentation, to determine a baseline set of the parameters (e.g., frequencies, phase shifts and/or amplification factors) of the transducer elements 230 to produce a focus at the target region. The controller 215 may also register the transducer elements 230 to images (e.g., patient preop images) obtained with the imager 112 or other modality. The initial positions of the transducer elements 230 may be random or may be based on anatomic information of the patient, e.g., air/tissue interfaces and bone volumes. The initial resulting focus may be improved using, e.g., the autofocusing approach described in PCT Appl. No. WO 2021/123906, filed on Dec. 18, 2020, the entire disclosure of which is hereby incorporated by reference. As described therein, by transmitting ultrasound waves to microbubbles at the target region and receiving reflections therefrom, the amplitudes and/or phases associated with the reflected ultrasound can be determined; based thereon, the transducer parameters (e.g., phase shifts and/or amplitudes) can be adjusted to compensate for aberrations caused, for example, by the skull. These reflections may be detected by operating at least some of the transducer elements 230 as receivers, using suitable (and conventional) circuitry deployed on the circuit board 230₂.

During this iterative focusing process, the controller 215 may compute candidate new positions for various ones of the transducer elements 230 and cause the individual transducer elements to assume these new positions (or to get as close thereto as possible) by signaling the associated motors to activate them and then deactivate them when, based on images recorded by the sensors 210 and the spatial mapping maintained by the controller 215, the transducer elements have reached the commanded positions. Alternatively, the controller 215 may issue instructions to a user via a mobile device (such as a smart phone or tablet) to guide manual transducer displacements. The controller 215 may operate the now-focused transducer array 205 to implement a treatment plan as described, for example, in U.S. Pat. No. 10,878,586, the entire disclosure of which is hereby incorporated by reference.

Other approaches to transducer tracking are possible. For example, instead of visible fiducials, the markers 235 may be electromagnetic sensors subject to excitation by a low-intensity field generator and communicating with a localization controller (as in the AURORA electromagnetic tracking system supplied by Northern Digital Inc., Ontario, Canada), or other suitable devices that may be detected and localized electromagnetically, optically, or otherwise by a suitable reader, or by MR coils. Moreover, for computational efficiency, the controller 215 may be configured to establish the absolute spatial location of a single marker 235 and then estimate the spatial locations of other markers relative to the already-localized marker (rather than establishing their absolute positions in the reference spatial coordinate system).

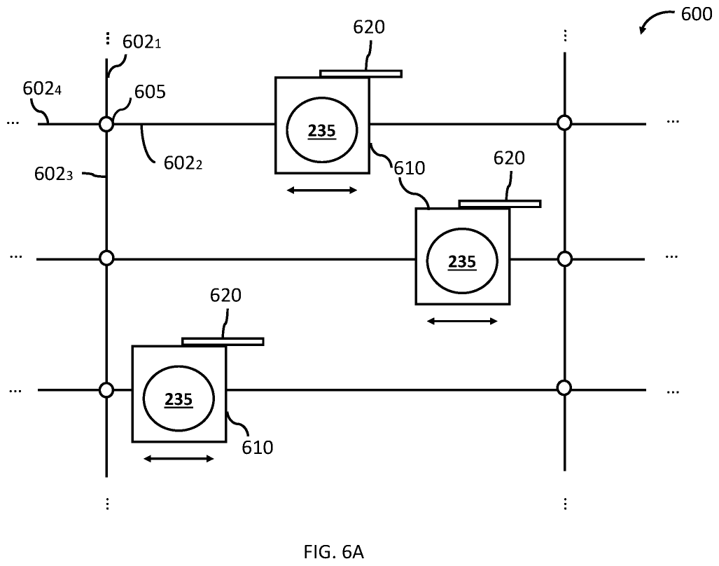

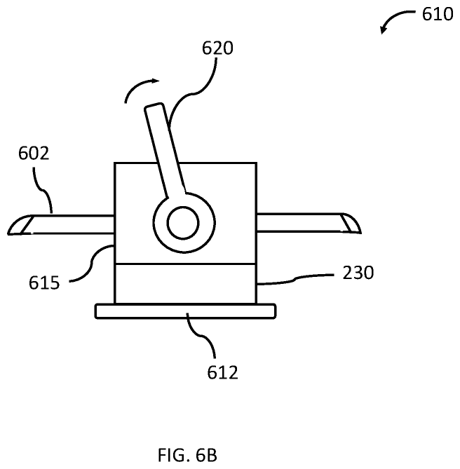

In another embodiment, illustrated in FIGS. 6A and 6B, the conformal scaffold is a two-dimensional grid 600 of linked frame segments running in perpendicular directions. The segments are hinged where connected to neighboring segments, allowing the scaffold 600 to behave as a conformal fabric. As an example, the segments 602₁ . . . 602₄ meet at a link 605 (which may be a simple interconnection of loops) that allows each of the segments 602 to have rotational freedom. Some (or, in some embodiments, all) of the segments 602 carry one or more transducer assemblies 610, which are capable of slidable movement therealong. As before, this movement may be manual or autonomous, e.g., using bidirectional motors, and the transducer assemblies 610 may feature visible markers 235. A manually adjustable transducer assembly 610 is illustrated in FIG. 6B. The transducer assembly 610 includes a transducer element 230 as described above, the bottom surface of which may be in contact with the patient's skin during use, i.e., with the conformal scaffold 600 draped over the patient's body or portion thereof. Alternatively, an acoustic coupling liquid such as water may be contained within a flexible container 612 beneath the transducer element 230.

The transducer element 230 is affixed to a slidable positioning member 615, which rides along the segment 602 and may be locked into position therealong using a locking mechanism 620. The transducer element 230 may also be pivotable, e.g., rotatable around the segment 602 prior to locking. For example, with the conformal scaffold 600 over a patient's chest, the transducer assemblies 610 may be manually positioned so as to lie between the patient's ribs and remain substantially perpendicular to the patient's skin. Alternatively or in addition, as described above, the positioning may be iterative and responsive to the controller 215 during a focusing procedure. If desired, the positioning member 615 may be configured to allow movement perpendicular to the segment 602 as well as along it.

In other embodiments, the scaffold 600 is rigid rather than conformal. For example, the scaffold 600 may be structured as a cage surrounding the patient or portion of the patient's anatomy in the bore of an MRI device. In such implementations, the joints 605 may be welds rather than links permitting movement.

In some embodiments, a system (e.g., the ultrasound system) may perform tracking and registration to an orientation of a target (e.g., a patient organ) relative to the transducer elements using a similar process and setup of tracking the transducer elements as described in FIG. 1-FIG. 6B.

As explained herein, in order to treat the target at a certain location in the body, all transmitting elements (e.g., transducer elements transmitting ultrasound waves) need to create constructive interference at a focus zone (e.g., a focus point). To achieve that the system (or a controller 215) may calculate a distance from the transmitting elements and the required target. However, the subject may move during the procedure, thus potentially causing the transmitting elements to misalign relative to the target. Thus, there is a need to be able to reorient the transmitting elements relative to the target under certain circumstances.

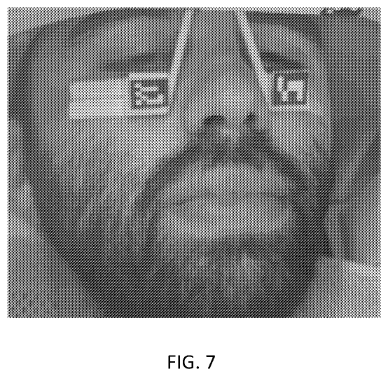

In these embodiments, one or more markers (e.g., markers 235) may be placed on a surface of the subject body. As the markers are placed on the skin of body, it is advantageous for the selected place on the body to have minimal movement of the skin to the body. For example, the bridge of the nose is less sensitive to movement (with respect to the skull) than the upper lip (with respect to the skull). FIG. 7 shows an example of two markers 235 being placed on a portion of the face that has minimal movement with respect to the skull (the upper cheeks, just below the eyes). In some embodiments, a marker can be referred to as a fiducial element or a visual fiducial. In some embodiments, instead of using one or more markers placed on the surface of the subject body, one or more body features (e.g., eyes, nose, mouth) may be used as “one or more markers.”

A CT/MRI image of the position/orientation of an identified target relative the one or more markers placed on the surface of the subject body may be captured using a CT or MRI device. Using the CT/MRI image, the distance from a target to the one or more markers may be calculated by the system using a computational processor.

Organ surface 3D data (e.g., a target surface 3D data) including the position of the one or more markers placed on the surface of the subject body may be collected. The organ surface 3D data point cloud (data) may represent geographical information and attribute information of the one or more markers placed on the surface of the subject body. The organ surface 3D data may be collected using one or more digital sensors. Examples of digital sensors include an IR/optical navigation/tracking device, a 3D camera, and an electromagnetic tracking device). The organ surface 3D data may be collected using the transducer elements contact points with the subject body.

The system, using a computational processor, may then register the one or more markers placed on the surface of the subject body to the target by comparing the CT/MRI image to the organ surface 3D data and matching the one or more markers in the CT/MRI image to the one or more markers in the organ surface 3D data.

The system using a computational processor, may then calculate a distance between the one or more markers placed on the surface of the subject body and the target.

In some embodiments, a 3D image including the position/orientation of the one or more markers placed on the surface of the subject body and the position/orientation of the markers corresponding to the transducer elements may be captured.

The system, using a computational processor, may calculate a distance between the one or more markers placed on the surface of the subject body and the one or more markers corresponding to the transducer elements.

The system, using a computational processor, may calculate a distance of the target relative to each of the transducer elements using (i) the distance between the one or more markers placed on the surface of the subject body and the one or more markers corresponding to the transducer elements and/or (ii) the distance between the one or more markers placed on the surface of the subject body and the target. Thus, even if the subject moves after registration of the target relative to the one or more markers placed on the surface of the subject body, the system may track the transducer elements and the one or more markers on the surface of the subject body to compensate for the movement after registration.

In general, the functionality of the controller 215 may be structured in one or more modules implemented in hardware, software, or a combination of both. For embodiments in which the functions are provided as one or more software programs, the programs may be written in any of a number of high-level languages such as PYTHON, JAVA, C, C++, C #, BASIC, various scripting languages, and/or HTML. Additionally, the software can be implemented in an assembly language directed to the microprocessor resident on a target computer (e.g., the controller); for example, the software may be implemented in Intel 80×8 6 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

In addition, the term “controller” used herein broadly includes all necessary hardware components and/or software modules utilized to perform any functionality as described above; the controller may include multiple hardware components and/or software modules and the functionality can be spread among different components and/or modules.

Certain embodiments of the present invention are described above. It is, however, expressly noted that the present invention is not limited to those embodiments; rather, additions and modifications to what is expressly described herein are also included within the scope of the invention.

1-50. (canceled)

51. A system for generating an ultrasound focus at a target region, the system comprising:
an ultrasound transducer array comprising a plurality of transducer elements movably positioned on a conformal scaffold;
one or more unique, transducer-specific visible fiducial elements respectively disposed over and identifying one or more corresponding transducer elements of the plurality of transducer elements and visible with the conformal scaffold positioned on a patient; and
a controller, operably coupled to the ultrasound transducer array, configured to:
acquire first images of the conformal scaffold from a plurality of sensors;
computationally establish, based on the first images, positions and orientations of the one or more fiducial elements with respect to the target region in a spatial coordinate system; and
based on the positions and orientations, operate at least some of the plurality of transducer elements to collectively transmit an ultrasound beam to the target region.

52. The system of claim 51, wherein the plurality of transducer elements and their corresponding fiducial elements are configured for mobility along the conformable scaffold.

53. The system of claim 51, wherein the controller is configured to represent the one or more fiducial elements and the target region in a common spatial reference frame.

54. The system of claim 51, wherein each of at least some of the fiducial elements is uniquely identifiable by the controller based on relative position, in the first images, with respect to (i) the conformal scaffold and/or (ii) at least one other fiducial element.

55. The system of claim 51, wherein each of the plurality of transducer elements has a corresponding fiducial element.

56. The system of claim 51, wherein the plurality of transducer elements include at least some transducer elements with corresponding fiducial elements and at least some transducer elements without corresponding fiducial elements, and the controller is configured to determine positions of the at least some of the transducer elements without corresponding fiducial elements from positions of the at least some of the transducer elements with corresponding fiducial elements.

57. The system of claim 51, wherein the one or more fiducial elements are 2D barcodes.

58. The system of claim 51, wherein the conformal scaffold includes a plurality of movable frame elements, at least some of the frame elements carrying at least some of the plurality of transducer elements.

59. The system of claim 51, wherein the controller is further configured to cause movement of the at least some of the plurality of transducer elements relative to the conformal scaffold to improve an ultrasound focus at the target region.

60. The system of claim 51, wherein each of the plurality of transducer elements comprises an emission element and a flexible container for a coupling liquid.

61. The system of claim 51, wherein the controller is further configured to:
obtain a patient scan of the target region relative to a plurality of body-specific visible fiducial elements positioned on a surface of the patient;
acquire second images of the body-specific visible fiducial elements relative to the one or more fiducial elements from the plurality of sensors; and
register the second images to the patient scan; and
determine a distance of each of the plurality of transducer elements relative to the target region using the registration of the acquired images to the patient scan.

62. The system of claim 61, wherein the controller is configured to register the second images to the patient scan by:
matching the plurality of body-specific visible fiducial elements in the patient scan to the plurality of body-specific visible fiducial elements in the second images.

63. The system of claim 61, wherein the controller is configured to determine the distance of each of the plurality of transducer elements relative to the target region by:
calculating a distance between the plurality of body-specific visible fiducial elements and the target region using the registration of the second images to the patient scan; and/or
calculating a distance between the plurality of body-specific visible fiducial elements and the one or more fiducial elements using the second images.

64. The system of claim 61, wherein the plurality of body-specific visible fiducial elements includes a unique anatomical body feature of the patient.

65. A method of generating an ultrasound focus at a target region using:
an ultrasound transducer array comprising a plurality of transducer elements movably positioned on a conformal scaffold;
one or more unique, transducer-specific visible fiducial elements respectively disposed over and identifying one or more corresponding transducer elements of the plurality of transducer elements and visible with the conformal scaffold positioned on a patient; and
a controller operably coupled to the ultrasound transducer array;
the method comprising the steps of operating the controller to:
acquire images of the conformal scaffold from a plurality of sensors;
computationally establish, based on the acquired images, positions and orientations of the one or more fiducial elements with respect to the target region in a spatial coordinate system; and
based on the positions and orientations, operate at least some of the plurality of transducer elements to collectively transmit an ultrasound beam to the target region.

66. The method of claim 65, further comprising operating the controller to identify each of at least some of the fiducial elements based on relative position, in the first images, with respect to (i) the conformal scaffold and/or (ii) at least one other fiducial element.

67. The method of claim 65, wherein the plurality of transducer elements include at least some transducer elements with corresponding fiducial elements and at least some transducer elements without corresponding fiducial elements, and the method further comprises operating the controller to determine positions of the at least some of the transducer elements without corresponding fiducial elements from positions of the at least some of the transducer elements with corresponding fiducial elements.

68. The method of claim 65, further comprising operating the controller to:
obtain a patient scan of the target region relative to a plurality of body-specific visible fiducial elements positioned on a surface of the patient;
acquire second images of the body-specific visible fiducial elements relative to the one or more fiducial elements from the plurality of sensors; and
register the second images to the patient scan; and
determine a distance of each of the plurality of transducer elements relative to the target region using the registration of the acquired images to the patient scan.

69. The method of claim 68, wherein operating the controller to register the second images to the patient scan comprises operating the controller to:
match the plurality of body-specific visible fiducial elements in the patient scan to the plurality of body-specific visible fiducial elements in the second images.

70. The method of claim 68, wherein operating the controller to determine the distance of each of the plurality of transducer elements relative to the target region comprises operating the controller to:
calculate a distance between the plurality of body-specific visible fiducial elements and the target region using the registration of the second images to the patient scan; and/or
calculate a distance between the plurality of body-specific visible fiducial elements and the one or more fiducial elements using the second images.
